(19) United States
(12) Patent Application Publication
(10) Pub. No.: US 2013/0338345 A1
(43) Pub. Date: Dec. 19, 2013

(54) Purification of Diphtheria Toxoid

(71) Applicants: Seshu K. Gudlavalleti, Omaha, NE (US); Jeeri R. Reddy, Omaha, NE (US)

(72) Inventors: Seshu K. Gudlavalleti, Omaha, NE (US); Jeeri R. Reddy, Omaha, NE (US)

(21) Appl. No.: 13/526,009

(22) Filed: Jun. 18, 2012

(51) Int. Cl. C07K 1/20

(52) U.S. Cl. 530/412

(57) ABSTRACT

The invention disclosed is a method of purifying Diphtheria Toxoid (DT) by Hydrophobic Interaction Chromatography (HIC). The chromatographic method of the present invention provides an effective removal of contaminating glycans present in carrier protein DT and thereby provides a highly purified form of carrier protein DT for the production or preparation of polysaccharide protein conjugate vaccines.

### BACKGROUND OF THE INVENTION

1. Field of the Invention

The present invention relates to a method for the purification of Diphtheria Toxoid (DT). In particular, the present invention relates to a chromatographic process for the removal of contaminating glycans from DT.

2. Description of the Related Art

Diphtheria Toxoid is used in DPT (Diphtheria, Pertussis, Tetanus) vaccines and is also one of the two most commonly used carrier proteins (the other being tetanus toxoid) in the preparation of polysaccharide protein conjugate vaccines.

### DT and Its Impurities in the Preparation of Conjugate Vaccines

Being detoxified, after purification by formaldehyde, DT contains traces of formaldehyde. Thimerosal (0.01%) is generally added as a preservative. Usually, these components are not acceptable in the final conjugate vaccine. In addition, toxoids isolated from Corynebacterium diphtheriae contain glycans as major contaminants (Murein (peptidoglycan, muropeptide) and arabinogalactan). Thimerosal and formaldehyde traces can be removed by simple dialysis. However, glycan impurities of larger molecular size are difficult to remove. These contaminating glycans interfere in the polysaccharide quantification which is very critical in vaccine formulations.

Therefore, there is a need for an invention to eliminate the short-comings in the prior art and to invent a method for the effective removal of glycans from carrier protein DT and thus provide a more purified form of DT for the production or preparation of polysaccharide protein conjugate vaccines.

### BRIEF SUMMARY OF THE INVENTION

The effective removal of glycans from DT becomes one of the essential steps in the preparation of polysaccharide protein conjugate vaccines. Hence, a method was developed to remove these contaminating hydrophilic glycans present in DT using Hydrophobic Interaction Chromatography (HIC).

The principle behind the present invention is that the hydrophilic glycans present in DT will flow-through with high ionic strength buffer [1.7 M (NH₄)₂SO₄] while hydrophobic DT will bind to the column matrix. Bound DT can then be eluted with low ionic strength [0.2 M (NH₄)₂SO₄] buffer.

Purified DT sample was then analyzed by HPAEC-PAD (High pH Anion Exchange Chromatography with Pulsed Amperometric Detection) for the presence of glycan and its composition. The results obtained were compared with the glycan content analysis results obtained for unpurified samples of DT (HPAEC-PAD results of DT sample before passing through HIC). This was done to estimate the percentage of purification efficiency of the developed process.

The results revealed that >87% of the contaminating glycans were removed from DT by the present invention.

Therefore, the main object of the present invention is to provide a method that will efficiently remove the glycans present as contaminants in carrier protein DT.

It is also an object of the present invention to provide a more purified form of carrier protein DT for the preparation of polysaccharide protein conjugate vaccines.

A further object of the present invention is to provide a scale up process that will efficiently remove glycans from DT used as a carrier protein in making conjugate vaccines.

### BRIEF DESCRIPTION OF THE DRAWINGS

FIG. 1: GC-MS profile on alditol acetate derivatized sample of DT to derive the glycan composition.

FIG. 2: HPAEC-PAD chromatogram on the analysis of glycans present in DT.

FIG. 3: HPAEC-PAD chromatogram on the analysis of glycan monosaccharides present in octyl sepharose column eluted DT.

FIG. 4: HPAEC-PAD chromatogram on the analysis of glycan removal by HIC column in a ten fold scale up process.

### DETAILED DESCRIPTION OF THE INVENTION

Glycans as known in the art and as used herein are polymeric sugars. Glycans can be oligomers or polymers of sugar residues, but typically contain at least three sugars, and can be linear or branched. A glycan may include natural sugar residues (e.g., glucose, N-acetylglucosamine, N-acetylneuraminic acid, galactose, mannose, fucose, hexose, arabinose, ribose, xylose, etc.) and/or modified sugars (e.g., 2′-fluororibose, 2′-deoxyribose, phosphomannose, 6′ sulfon-acetylglucosamine, etc.). The term ‘glycan’ includes homo and heteropolymers of sugar residues. The term ‘glycan’ also encompasses a glycan component of a glycoconjugate (e.g. glycoprotein, glycolipid, proteoglycan, etc.). The term also encompasses free glycans, including glycans that have been cleaved or otherwise released from a glycoconjugate.

### Analysis of Glycan Content of Carrier Protein DT Before and After Dialysis

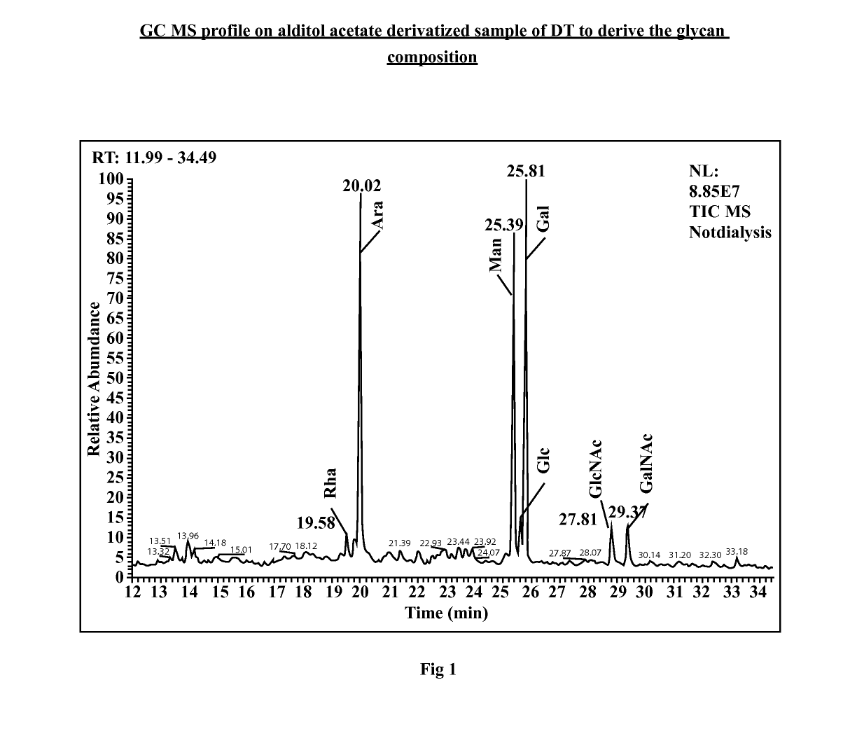

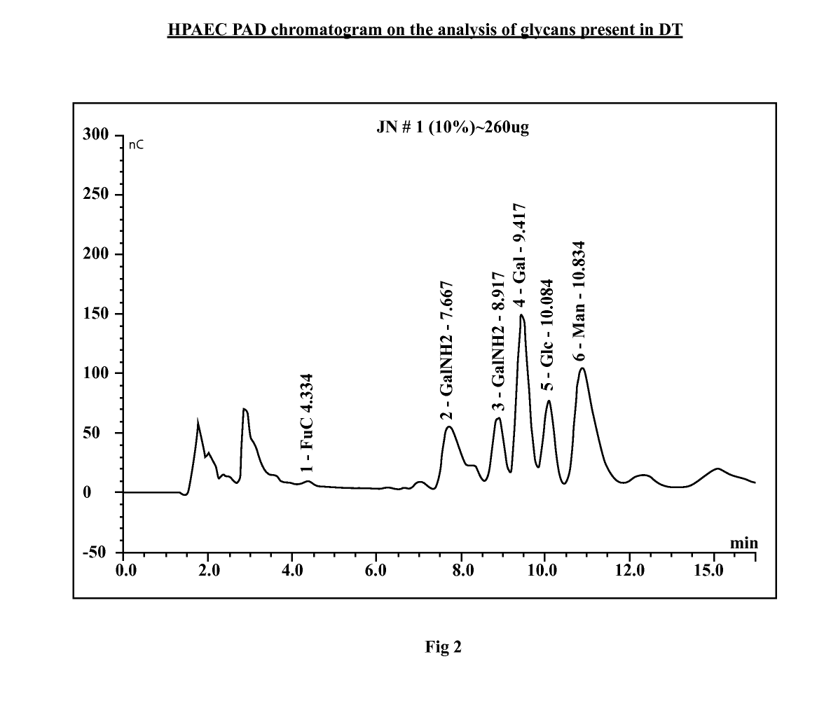

As a measure of understanding the nature and composition of glycans present in carrier protein DT, samples of DT before and after dialysis (25 KDa MWCO dialysis) were analyzed (at Glycotechnology Core facility, University of California, San Diego) by Gas Chromatography-Mass Spectrometry (GC-MS) (FIG. 1) and HPAEC-PAD (FIG. 2) for monomeric sugar composition.

The results revealed that DT contained 0.6% (w/v) of glycan with a monosaccharide composition of arabinose, fucose, galactosamine, glucosamine, galactose, glucose and mannose. Mannose was the most abundant sugar present in the contaminating glycan followed by galactose and arabinose. Further, dialysis removed only 6% of the total glycans contaminating DT. Also, the sugar composition indicated that the contaminating glycans of DT constitute C. diphtheriae surface polysaccharide and possibly Lipoarabinomannan (LAM) like lipo-oligos.

In the present invention, the contaminating glycans of carrier protein DT were removed by Hydrophobic Interaction Chromatography (HIC). The hydrophilic glycans present in DT will flow-through with high ionic strength buffer [1.7 M (NH₄)₂SO₄] while hydrophobic DT will bind to the column matrix. Bound DT can then be eluted with low ionic strength [0.2 M (NH₄)₂SO₄] buffer.

The efficiency of glycan removal of the present invention was then calculated by comparing the glycan content of the protein sample (DT) before loading onto the column to that of the glycan content of the eluted protein. The glycan content of the protein samples were determined by GC-MS and HPAEC-PAD.

Removal of Glycan from DT

### Example I

### Hydrophobic Interaction Chromatographic Removal of Glycans

The matrix used for this process was Octyl sepharose 4 Fast Flow purchased from GE Healthcare. It was HIC media for bioprocess separation having cross-linked, 4% agarose derivative containing octyl ligand —R—O—CH₂—CH(OH)—CH₂—O—(CH₂)₇—CH₃.

50×0.7 cm Econopack GE glass column was packed with Octyl sepharose 4 Fast Flow in binding buffer and equilibrated with the same buffer. The column flow rate was adjusted to 1.0 ml/minute by gravity. The binding buffer was 1.7 M (NH₄)₂SO₄ with 50 mM Tris of pH 7.5. Similarly the elution buffer was 0.2 M (NH₄)₂SO₄ with 50 mM Tris of pH 7.5.

One milliliter of DT sample having a concentration of 10 mg/ml was mixed with 1 ml of binding buffer [1.7 M (NH₄)₂SO₄] (1:1 ratio) to form the sample mixture. From this 2 m1 of the sample mixture, 250 μl was taken and kept separately for glycan analysis (to determine sugar composition of the contaminating glycans in the DT sample before loading on to the HIC column). The remaining 1.75 ml aliquot of the sample mixture (DT and binding buffer 1:1 mix) was placed or loaded onto the surface of the column (41 cm bed height and 17 ml bed volume) and was allowed to flow at the rate of 1 ml/min. After the sample has completely entered the bed, 1.0 ml of binding buffer was placed on top of it and allowed to enter. A buffer reservoir with binding buffer was connected to the top of the column and allowed to flow at the same flow rate of 1.0 ml/min. Two column bed volumes (2>17=34 ml) of Flow-Through (FT) were collected. The binding buffer in the reservoir was then replaced with elution buffer and the bound protein was eluted with two column bed volumes.

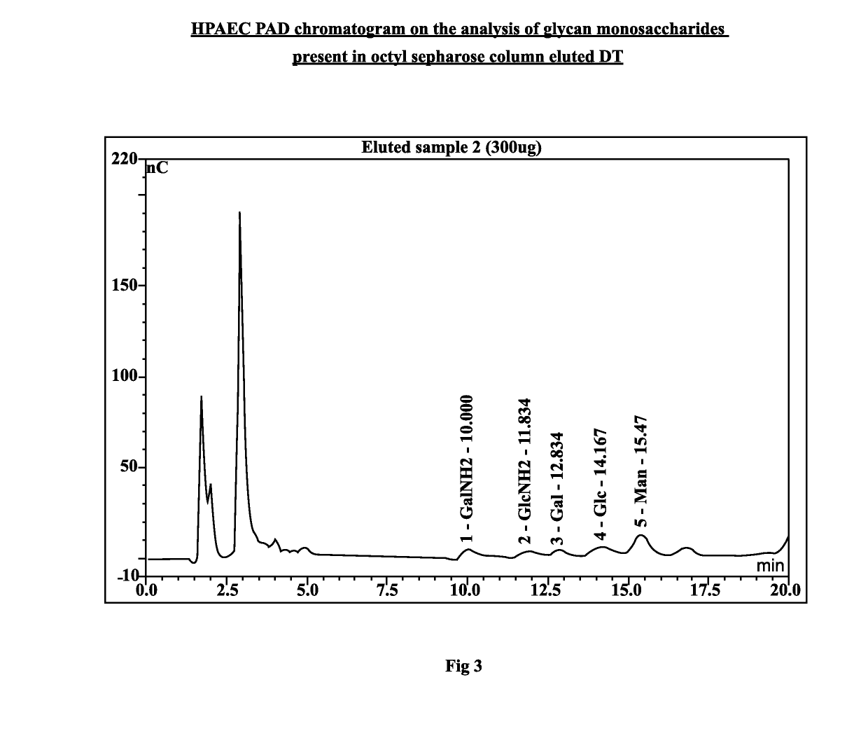

Both the Flow-Through and eluted volumes were concentrated 10 to 15 folds separately using 3000 MW cut off spin filters. The three samples: (1) starting material (DT before passing through HIC); (2) Flow-Through concentrate; and (3) elute concentrate (DT after passing through HIC) were analyzed for glycan composition by GC-MS and HPAEC-PAD to determine the efficiency of the present invention. FIG. 3 shows the results of glycan monosaccharide analysis of octyl sepharose column eluted DT.

### Results

TABLE 1
Analysis of Diphtheria Toxoid (DT) using HPAEC-PAD before and after passing through
the octyl sepharose (HIC) column for removal of contaminating glycans

| | | | | | | | | | |
|---|---|---|---|---|---|---|---|---|---|
| | | | | | | | | | Normalized |
| | | | | | | | | | Values for |
| | Protein | | | | | | | Total | Sample |
| Sample name | Content | Fuc | GalNH₂ | GlcNH₂ | Gal | Glc | Man | Carb | Vol |

% (μg/100 μg sample):

| | | | | | | | | | |
|---|---|---|---|---|---|---|---|---|---|
| DT Before | 600 ug | 0.000 | 0.051 | 0.056 | 0.016 | 0.050 | 0.000 | 0.17 | 0.4675 |
| (8 mg/ml) | (75 μl) | | | | | | | | |
| Flow through | 75 μl | 0.000 | 1.087 | 0.781 | 0.000 | 0.000 | 0.000 | 1.87 | N/A |
| (200 μl) | | | | | | | | | |
| DT Eluted | 1.65 mg | 0.000 | 0.010 | 0.006 | 0.004 | 0.008 | 0.033 | 0.06 | 0.0600 |
| (22 mg/ml) | (75 μl) | | | | | | | | |

Elution buffer: 0.2M (NH₄)₂SO₄
Binding buffer: 1.7M (NH₄)₂SO₄

The efficiency of glycan removal of the present invention was calculated by comparing the glycan content of the protein sample before loading onto the column to that of the glycan content of the eluted protein. The results showed that the Hydrophobic Interaction Chromatography of the present invention removed >87% of the glycans contaminating the carrier protein DT.

### Example II

### Ten Fold Scale Up on Glycan Removal

GLP (Good Laboratory Practices) scale hydrophobic interaction column chromatography in the glycan removal process (Example I) was also attempted with ten fold scaling up amounts of DT to be purified.

A 50×2.5 cm Econopack GE glass column and Octyl Sepharose 4 Fast flow (from GE) media were used for this purpose. The column was packed in binding buffer and equilibrated with the same buffer. The binding and the elution buffers were same as that used for small scale glycan removal process (Example I).

10 ml of 10 mg/ml DT was mixed with 10 ml of binding buffer (1:1 ratio) and placed or loaded on the octyl sepharose column (40.7 cm bed height and 200 ml bed volume). Gravity flow was used to achieve a column flow rate of 2.5 ml/min. Two bed volumes (400 ml) of Flow-Through and two bed volumes (400 ml) of elutions were collected.

40 ml representative volumes of Flow-Through and elutions were separately concentrated 20 folds using 3K MWCO Millipore spin filters. The three samples of representative starting material, Flow-Through concentrate and elute concentrate were tested for their glycan contents and concentration by HPAEC-PAD and GC-MS to see the efficiency of the scale up process.

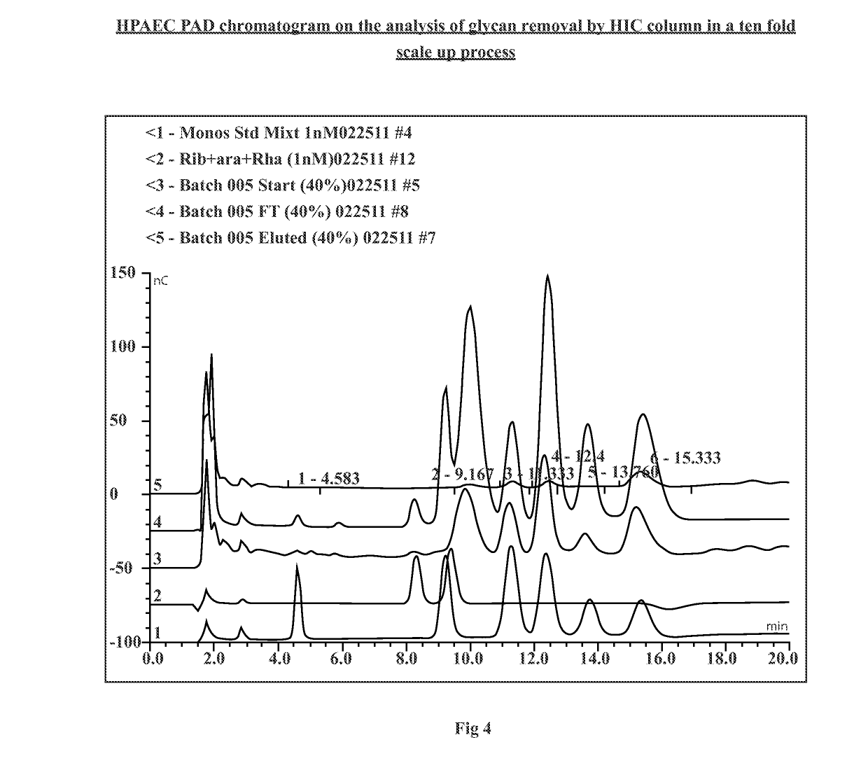

FIG. 4 shows the results of HPAEC-PAD chromatogram analysis of a ten fold scale up process on the removal of glycans by HIC (200 ml bed volume) column. The eluted fraction represented by number 5 in FIG. 4 indicates the glycan removal.

The small scale (Example I) and scale up (Example II) processes indicate that this technology is applicable to GMP (Good Manufacturing Practices) conditions for efficient removal of contaminating glycans from DT used as carrier protein in making conjugate vaccines.

What is claimed is:

1. A method of purification of diphtheria toxoid comprising:
(a) loading a sample of diphtheria toxoid containing glycan as an impurity into a hydrophobic interaction chromatographic column, equilibrated with binding buffer which helps the diphtheria toxoid in binding to said column matrix;
(b) allowing the sample to flow-through the column matrix;
(c) washing the column matrix with two column bed volumes of binding buffer; and
(d) eluting the bound diphtheria toxoid with two column bed volumes of elution buffer; whereby, the glycan content in the purified diphtheria toxoid is reduced.

2. A method according to claim 1, wherein the hydrophobic interaction chromatography matrix is selected from the group consisting of resins substituted with octyl ligand.

3. A method according to claim 1, wherein the binding buffer is 1.7M ammonium sulphate with 50 mM tris.

4. A method according to claim 1, wherein the elution buffer is 0.2M ammonium sulphate with 50 mM tris.

5. A method according to claim 1, wherein the pH of the binding and elution buffers is 7.5.

6. A method according to claim 1, wherein the sample is diphtheria toxoid having a concentration of 10 mg/ml.

7. A method according to claim 1, wherein the flow rate of the column is achieved by gravity flow.

8. A method according to claim 1, wherein the glycan content in the purified diphtheria toxoid is reduced by at least 87%.
